(19) United States
(12) Patent Application Publication
(10) Pub. No.: US 2024/0212821 A1
(43) Pub. Date: Jun. 27, 2024

(54) ELECTRONIC APPARATUS AND CONTROL METHOD FOR AN ELECTRONIC APPARATUS

(71) Applicant: Toshiba Tec Kabushiki Kaisha, Tokyo (JP)

(72) Inventor: Takaho WATANABE, Mishima Shizuoka (JP)

(21) Appl. No.: 18/449,975

(22) Filed: Aug. 15, 2023

(30) Foreign Application Priority Data
Dec. 23, 2022 (JP) 2022-207130

(51) Int. Cl. G16H 20/30; G06V 10/70; G06V 40/20; G16H 40/63

(52) U.S. Cl. CPC G16H 20/30; G06V 10/70; G06V 40/23; G16H 40/63

(57) ABSTRACT

According to an embodiment, an electronic apparatus detects a posture of a user doing an exercise. The electronic apparatus counts the number of repetitions a determined exercise has been repeated on the basis of the posture of the user. In addition, the electronic apparatus authenticates the user under a condition that the number of repetitions of the determined exercise has reached a set number of repetitions equal to or larger than 2.

### CROSS-REFERENCE TO RELATED APPLICATIONS

This application is based upon and claims the benefit of priority from the prior Japanese Patent Application No. 2022-207130, filed on Dec. 23, 2022, the entire contents of which are incorporated herein by reference.

### FIELD

Embodiments of the present invention relate to an electronic apparatus that performs user authentication and a control method for an electronic apparatus.

### BACKGROUND

Some electronic apparatuses require user authentication for using their functions. Three methods for user authentication as follows are generally known. The first method is a method of inputting information that only the user knows, such as password and personal identification number (PIN) code. The second method is a method of using a tool such as ID card and token for generating a one-time password. The third method is a method of using biometric information such as fingerprint, iris, face, and veins of the user. However, all the methods apply no physical load on the user.

By the way, people who works at their desk have been increasing in recent years. More and more people have health problems such as waist pain, neck pain, and eye pain because of their continuous work at the same posture. Daily proper exercises can reduce such health problems. Therefore, the use of an exercise for user authentication will reduce health problems such as waist pain, neck pain, and eye pain because it forces the user to habitually exercise for user authentication.

### BRIEF DESCRIPTION OF THE DRAWINGS

FIG. 1 is a schematic view showing the outer appearance of a smartphone according to a first embodiment.

FIG. 2 is a block diagram showing a main hardware configuration of the smartphone according to the first embodiment.

FIG. 3 is a diagram showing a configuration of an exercise-specific determination model according to the first embodiment.

FIG. 4 is a diagram showing a configuration of a setting table according to the first embodiment.

FIG. 5 is a flowchart showing information processing when a processor of the smartphone according to the first embodiment operates on a setting mode of a user authentication APL.

FIG. 6 is a flowchart showing information processing when the processor of the smartphone according to the first embodiment operates on an authentication mode of the user authentication APL.

FIG. 7 is a schematic view showing a display example of a setting screen according to the first embodiment.

FIG. 8 is a schematic view showing a display example of a confirmation screen according to the first embodiment.

FIG. 9 is a schematic view showing the outer appearance of a multifunction peripheral according to a second embodiment.

FIG. 10 is a block diagram showing a main hardware configuration of a multifunction peripheral (MFP) according to a second embodiment.

FIG. 11 is a diagram showing a configuration of a per-user setting table according to the second embodiment.

FIG. 12 is a flowchart showing information processing when a processor of the MFP according to the second embodiment operates on a setting mode of a user authentication APL.

FIG. 13 is a flowchart showing information processing when the processor of the MFP according to the second embodiment operates on an authentication mode of the user authentication APL.

### DETAILED DESCRIPTION

In accordance with one embodiment, an electronic apparatus that requires authentication of a user includes a camera, a storage, and a processor. The camera images the user. The storage stores a determination model. The determination model is a machine learning algorithm that receives input of a moving image of the user captured by the camera, detects parts of a body of the user on the basis of the moving image, and determines an exercise of the user on the basis of movements of the parts. The processor loads the moving image captured by the camera. The processor detects a posture of the user doing the exercise by inputting the loaded captured moving image to the determination model and obtaining a determination result. The processor counts the number of repetitions a pre-set exercise has been repeated on the basis of the detected posture of the user. In addition, the processor authenticates the user under a condition that the counted number of repetitions has reached the set number of repetitions.

Hereinafter, embodiments of an electronic apparatus that enables the user to habitually do proper exercises for user authentication, a control method for an electronic apparatus, and a program therefor will be described with reference to the drawings.

In the drawings, the same reference signs denote the same or similar portions.

### First Embodiment

A first embodiment uses a smartphone as an example of an electronic apparatus that requires user authentication for using its function. Various types of application software can be installed in the smartphone. In view of this, in the present embodiment, a folder that can be unlocked only when a user has been authenticated is prepared in the smartphone. Then, the user is authenticated by exercising in the front of the smartphone, the folder is unlocked, and the user is allowed to use the application software. In this manner, the user can habitually do proper exercises for user authentication.

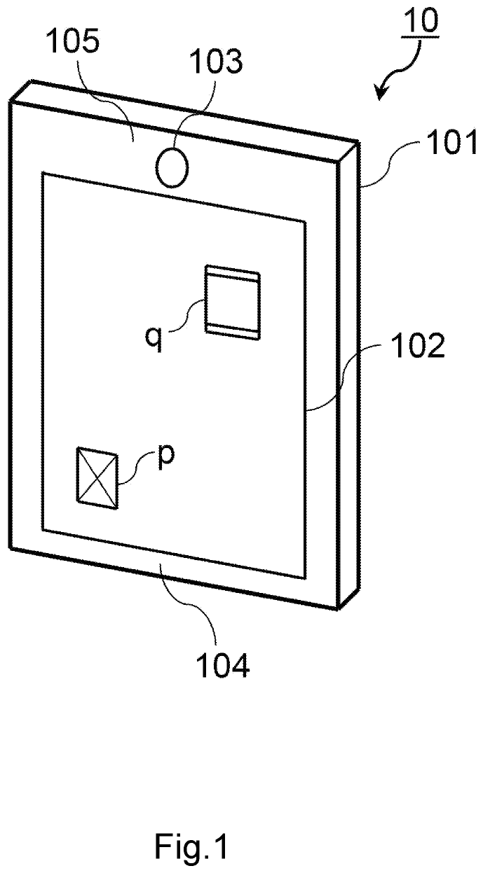

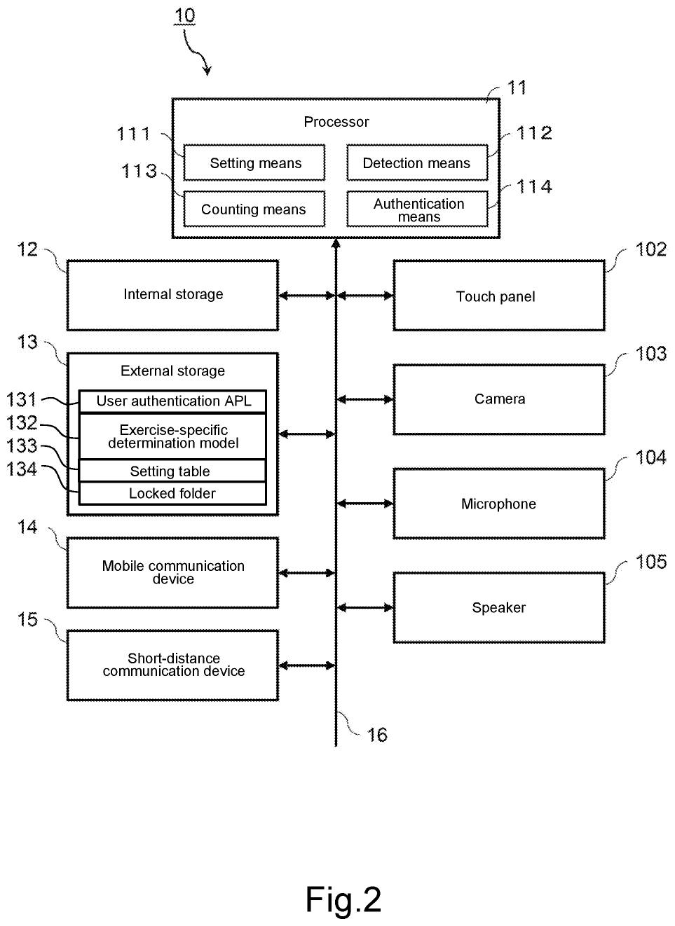

FIG. 1 is a schematic view showing the outer appearance of a smartphone 10. FIG. 2 is a block diagram showing a main hardware configuration of the smartphone 10. As shown in FIG. 1, the smartphone 10 has a touch panel 102 at a front portion of a main body 101. The touch panel 102 is a user interface that serves both as a display device and an input device. A user of the smartphone 10 operates the touch panel 102 and inputs various types of information or gets various types of information from the screen of the touch panel 102.

Moreover, the smartphone 10 includes a camera 103, a microphone 104, a speaker 105, for example, in the main body 101.

The camera 103 is an image capturing device. For example, a CCD camera is used as the camera 103. The camera 103 captures a still image or moving image in an imaging area, which is a front or back side of the main body 101. The microphone 104 is an input device for sound or voice. A universal sound collecting microphone is used as the microphone 104. The speaker 105 is an output device for sound or voice. A universal loudspeaker is used as the speaker 105.

As shown in FIG. 2, the smartphone 10 includes a processor 11, an internal storage 12, an external storage 13, a mobile communication device 14, and a short-distance communication device 15. The processor 11, the internal storage 12, the external storage 13, the mobile communication device 14, and the short-distance communication device 15 are connected to one another through a system communication channel 16 in the smartphone 10. The system communication channel 16 includes an address bus, a data bus, a control signal line, for example. The smartphone 10 configures a computer by connecting the processor 11, the internal storage 12, the external storage 13, the mobile communication device 14, and the short-distance communication device 15 to one another through the system communication channel 16. Then, the smartphone 10 connects the touch panel 102, the camera 103, the microphone 104, and the speaker 105 to the computer via the system communication channel 16. It is needless to say that devices provided in the smartphone 10 are not limited to the touch panel 102, the camera 103, the microphone 104, and the speaker 105.

The processor 11 corresponds to a central processing portion of the computer. The processor 11 controls the respective units to achieve various functions as the smartphone 10 in accordance with an operating system or application software. The processor 11 is, for example, a central processing unit (CPU).

The internal storage 12 corresponds to a main storage portion of the above-mentioned computer. The internal storage 12 includes a nonvolatile memory area and a volatile memory area. The internal storage 12 stores an operating system or application software in a nonvolatile memory area. The internal storage 12 is capable of storing data required for the processor 11 to execute processing for controlling the respective units in a nonvolatile or volatile memory area. The volatile memory area of the internal storage 12 is used as a work area where processor 11 rewrites data as appropriate. The non-volatile memory area is, for example, a read only memory (ROM). The volatile memory area is, for example, a random access memory (RAM).

The external storage 13 corresponds to an auxiliary storage portion of the above-mentioned computer. For example, a memory card such as an SD card or a USB memory can be the external storage 13. The external storage 13 stores data used for the processor 11 to execute various types of processing, data generated by processing of the processor 11, and the like. The external storage 13 is capable of storing the above-mentioned application software.

The mobile communication device 14 is an interface for performing data communication with an external apparatus via a mobile communication network. The mobile communication device 14 employs wireless communication standards, for example, Wi-Fi (registered trademark) for connecting the mobile communication network.

The short-distance communication device 15 is an interface for performing data communication with a wireless communication medium by near-field communication. The short-distance communication device 15 employs wireless communication standards, for example, near field communication (NFC) or Bluetooth (registered trademark) for performing data communication with the wireless communication medium.

In the present embodiment, a user authentication APL 131 is installed in the smartphone 10 having the above-mentioned configuration. The user authentication APL 131 is application software for user authentication. The user authentication APL 131 may be installed in the internal storage 12 or may be installed in the external storage 13. FIG. 2 shows a case where the user authentication APL 131 is installed in the external storage 13. A method of installing the user authentication APL 131 in the smartphone 10 is not particularly limited. The user authentication APL 131 can be installed in the internal storage 12 or the external storage 13 of the smartphone 10 by recording the user authentication APL 131 on a removable recording medium or delivering the user authentication APL 131 by communication via the mobile communication network. The recording medium can take any form, e.g., a CD-ROM or memory card as long as the recording medium can store a program and a device can read the program from recording medium.

The user authentication APL 131 is application software for detecting a posture of the user doing an exercise from an image captured by the camera 103, counting the number of repetitions the determined exercise has been repeated on the basis of the posture of the user, and authenticating the user of the smartphone 10 under the condition that the number of repetitions has reached a set number of repetitions equal to or larger than 2. When the user authentication APL 131 has authenticated the user, the folder is unlocked in the smartphone 10 and the application software stored in the folder is allowed to be used.

In order to achieve such functions, the user authentication APL 131 is installed in the smartphone 10, such that storage areas of an exercise-specific determination model 132, a setting table 133, and a locked folder 134 are formed in the external storage 13.

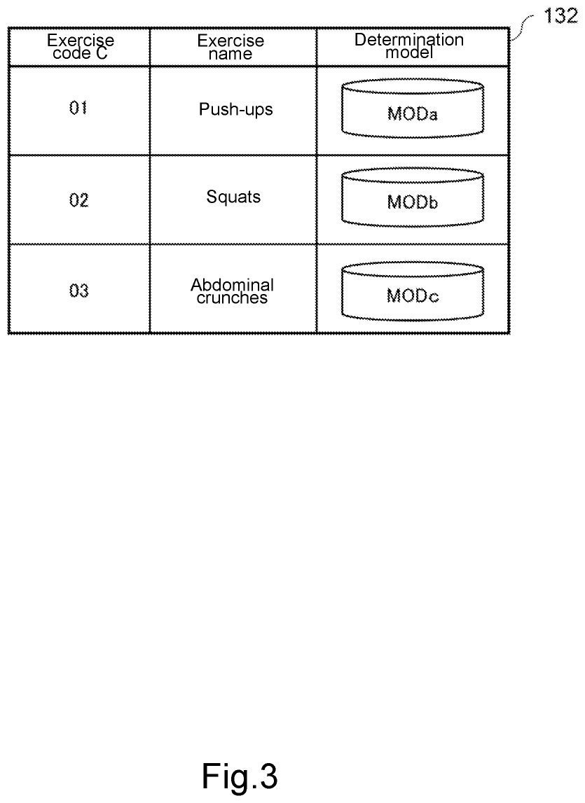

The exercise-specific determination model 132 is an area in which an exercise name and a determination model are described for each exercise code C for identifying an exercise used for user authentication as shown in FIG. 3. In the present embodiment, the exercises are three exercises “push-ups”, “squats”, and “abdominal crunches”. The determination model is a machine learning algorithm that receives the input of a moving image of the user captured by the camera 103, detects parts of the user, such as head, shoulders, elbows, wrists, waist, knees, and ankles, from the moving image, and recognizes an exercise of the user on the basis of movements of the parts. Since such a technology is well-known as a posture estimation technology such as OpenPose, its detailed description is omitted here.

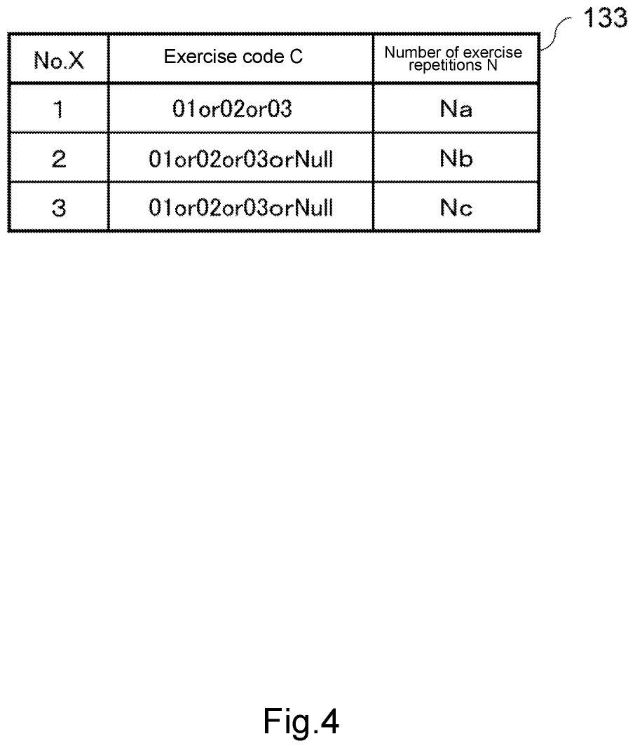

The setting table 133 is an area for setting the exercise code C and the number of exercise repetitions N in association with the table number No. X as shown in FIG. 4. Then, any one of the exercise code “01” of the exercise “push-ups”, the exercise code “02” of the exercise “squats”, or the exercise code “03” of the exercise “abdominal crunches” is set in the field of the exercise code C of the table number No. 1. “Null” representing no settings besides the exercise codes “01”, “02”, and “03” can be set in the field of the exercise code C of the table number No. 2 and so on. On the other hand, as to the number of exercise repetitions N, integers equal to or larger than “2” are set with respect to the table numbers Nos. X where the exercise codes “01”, “02”, and “03” have been set. The number of exercise repetitions N is “0” with respect to the table number No. X where “Null” has been set as the exercise code C.

The locked folder 134 is a folder that is unlocked in a case where the user authentication APL 131 has authenticated the user. An icon p of the user authentication APL 131 and an icon q of the locked folder 134 are displayed on the screen of the touch panel 102 as shown in FIG. 1 by installing the user authentication APL 131 in the smartphone 10. For example, the user drag and drops an icon of arbitrary application software excluding the user authentication APL 131 to the icon q in order to store the application software in the locked folder 134.

Moreover, the user can start the user authentication APL 131 by making a touch operation on the icon p. When the user authentication APL 131 is started, the processor 11 achieves functions as a setting means 111, a detecting means 112, a counting means 113, and an authentication means 114 as shown in FIG. 2.

The setting means 111 is a function of setting to the setting table 133 an arbitrary exercise code C and the number of exercise repetitions N in the order of the table numbers Nos. X (FIG. 4). The detecting means 112 is a function of detecting a posture of the user doing an exercise in the front of the smartphone 10 by using the exercise-specific determination model 132 (FIG. 3). The counting means 113 is a function of counting the number of repetitions the determined exercise has been repeated on the basis of the posture of the user. The authentication means 114 is a function of authenticating the user under the condition that the number of repetitions of the determined exercise has reached a set number of repetitions equal to or larger than 2.

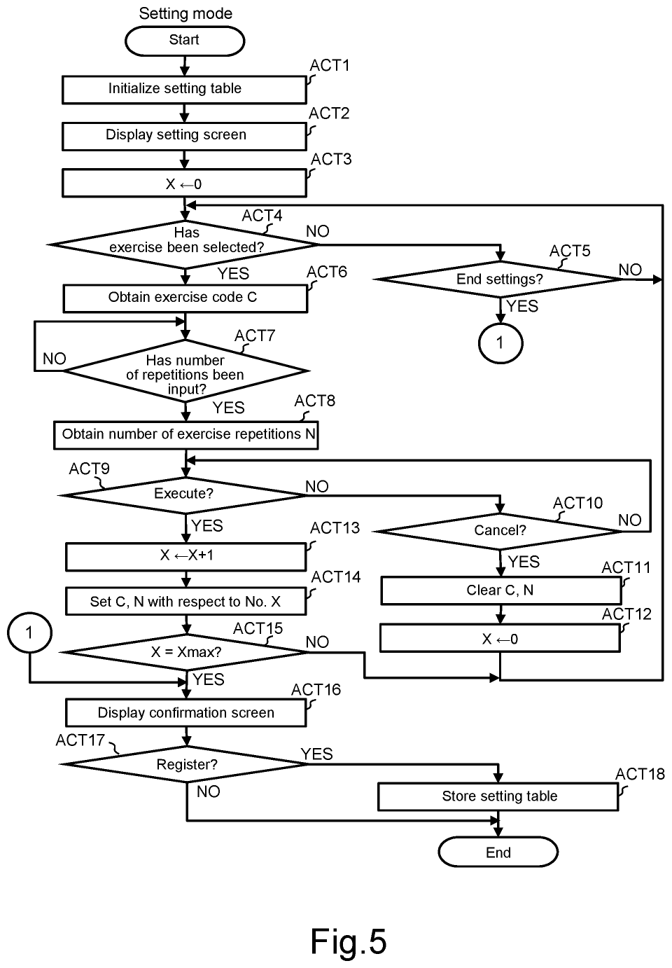

The setting means 111 is a function achieved by the processor 11 operating on a setting mode of the user authentication APL 131. The detecting means 112, the counting means 113, and the authentication means 114 are functions achieved by the processor 11 operating on an authentication mode of the user authentication APL 131. Next, principal operations of the processor 11 will be described with reference to FIGS. 5 to 8.

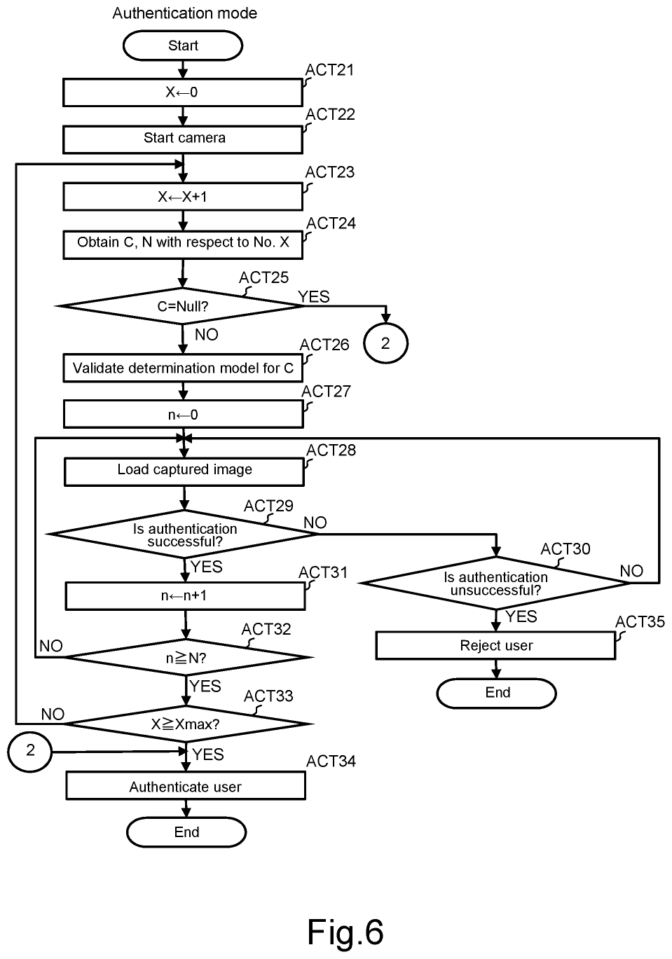

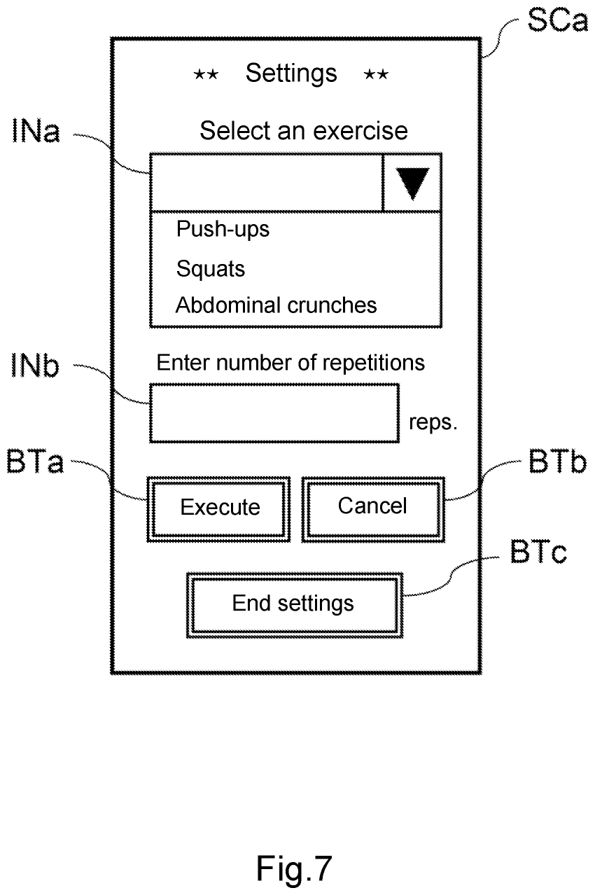

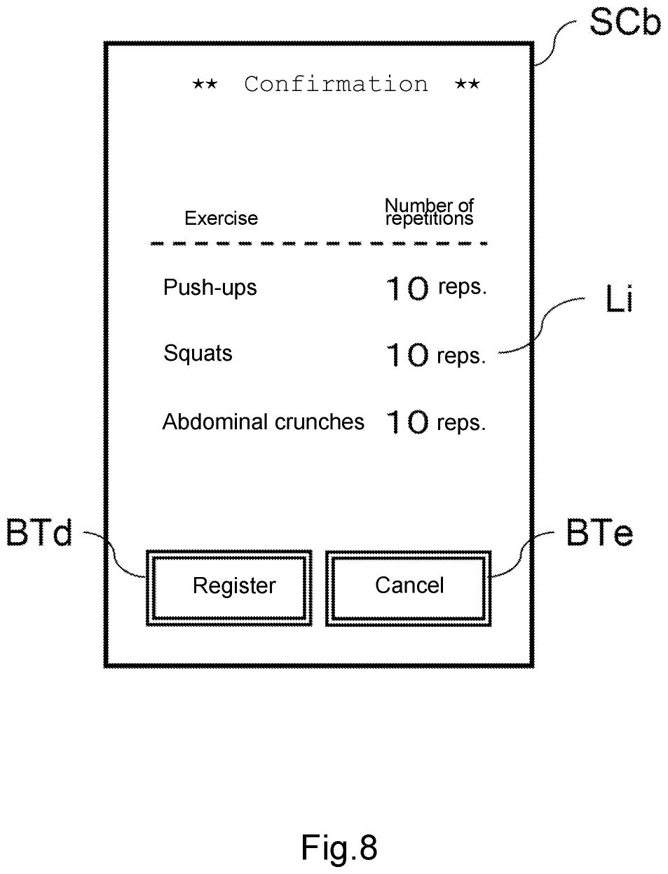

FIG. 5 is a flowchart showing information processing when the processor 11 operates on the setting mode of the user authentication APL 131. FIG. 6 is a flowchart showing information processing when the processor 11 operates on the authentication mode of the user authentication APL 131. FIG. 7 is a schematic diagram showing a setting screen SCa displayed on the touch panel 102. FIG. 8 is a schematic diagram showing a confirmation screen SCb displayed on the touch panel 102. It should be noted that the procedure of the information processing shown in each flowchart is an example. The procedure can be modified as appropriate as long as it can provide similar effects. Moreover, the setting screen SCa and the confirmation screen SCb are also examples. Screen lay-out, displayed text, and the like can be modified as appropriate as long as they can provide similar information to the user.

The user of the smartphone 10 starts the user authentication APL 131 by touching the icon p displayed on the touch panel 102. Then, a menu screen of the user authentication APL 131 is displayed on the touch panel 102. Next, the user selects a setting mode from the menu screen. Then, the processor 11 starts the information processing of the procedure shown in the flowchart of FIG. 5.

In ACT 1, the processor 11 initializes the setting table 133 (FIG. 4). This initialization clears the exercise code C and the number of exercise repetitions N described in association with each table number No. X in the setting table 133 (FIG. 4). As a result, the exercise codes C are “Null”. The numbers of exercise repetitions N are “0”.

The processing of the processor 11 that has initialized the setting table 133 shifts to ACT 2. In ACT 2, the processor 11 switches the screen of the touch panel 102 to the setting screen SCa (FIG. 7). As shown in FIG. 7, a pull-down box INa for selecting one of the exercises and an input box INb of the number of exercise repetitions N are arranged on the setting screen SCa. Moreover, software keys of an “execute” button BTa, a “cancel” button BTb, and an “end settings” button BTc are arranged on the setting screen SCa.

The user checks the setting screen SCa and selects one exercise from the exercises “push-ups”, “squats”, and “abdominal crunches” displayed on the pull-down box INa. Then, the user inputs the number of repetitions equal to or larger than “2” by using a ten key displayed when the user touches the input box INb.

For example, in a case where settings are made so that the user can be authenticated by doing “push-ups” five times, the user selects the exercise “push-ups” through the pull-down box INa and inputs the number of repetitions “5” to the input box INb. Then, the user inputs the “execute” button BTa and also inputs the “end settings” button BTc.

For example, in a case where settings are made so that the user can be authenticated by doing “push-ups” five times and also doing “squats” five times, the user first selects the exercise “push-ups” through the pull-down box INa and inputs the number of repetitions “5” to the input box INb. Subsequently, the user selects the exercise “squats” through the pull-down box INa and inputs the number of repetitions “5” to the input box INb. Then, the user inputs the “execute” button BTa and also inputs the “end settings” button BTc. In this regard, the same applies to a case where three exercises are selected. It should be noted that in order to redo the selection of the exercise or the input of the number of repetitions, the user only needs to input the “cancel” button BTb before inputting the “end settings” button BTc.

The description to FIG. 5 will be continued. In ACT 3, the processor 11 that has displayed the setting screen SCa resets a number counter X to “0”. Then, in ACT 4, the processor 11 stands by until an exercise is selected. In the stand-by state, in ACT 5, the processor 11 checks whether or not the “end settings” button BTc has been input. In a case where the “end settings” button BTc has been input (ACT 5: YES), the processing of the processor 11 shifts to ACT 16 from ACT 5. The processing after ACT 16 will be described later.

In a case where the “end settings” button BTc has not been inputted (ACT 5: NO) and any one of the exercises has been selected through the pull-down box INa (ACT 4: YES), the processing of the processor 11 shifts to ACT 6 from ACT 4. In ACT 6, the processor 11 obtains an exercise code C of the exercise selected through the pull-down box INa. For example, in a case where the exercise “push-ups” has been selected, the processor 11 obtains “01” as the exercise code C. For example, in a case where the exercise “squats” has been selected, the processor 11 obtains “02” as the exercise code C. For example, in a case where the exercise “abdominal crunches” has been selected, the processor 11 obtains “03” as the exercise code C.

The processing of the processor 11 that has obtained the exercise code C shifts to ACT 7. In ACT 7, the processor 11 stands by until the number of repetitions is input to the input box INb. The number of repetitions is an integer equal to or larger than 2. In a case where the number of repetitions is input to the input box INb (ACT 7: YES), the processing of the processor 11 shifts to ACT 8 from ACT 7. In ACT 8, the processor 11 obtains the number of repetitions input to the input box INb as the number of exercise repetitions N.

The processing of the processor 11 that has obtained the number of exercise repetitions N shifts to ACT 9 from ACT 8. In ACT 9, the processor 11 checks whether or not the “execute” button BTa has been input. In a case where the “execute” button BTa has not been input (ACT 9: NO), the processing of the processor 11 shifts to ACT 10. In ACT 10, the processor 11 checks whether or not the “cancel” button BTb has been input. In a case where the “cancel” button BTb has not been input (ACT 10: NO), the processing of the processor 11 returns to ACT 9. In this manner, the processor 11 stands by until the “execute” button BTa is input or the “cancel” button BTb is input as ACT 9 and ACT 10.

In a case where the “cancel” button BTb has been input (ACT 10: YES) in the stand-by state in ACT 9 and ACT 10, the processing of the processor 11 shifts to ACT 11 from ACT 10. In ACT 11, the processor 11 clears the obtained exercise code C and the number of exercise repetitions N. Moreover, in ACT 12, the processor 11 resets the number counter X to “0”. Then, the processing of the processor 11 returns to ACT 4. Then, the processor 11 executes the processing after ACT 4 in a manner similar to that described above.

In a case where the “execute” button BTa has been input (ACT 9: YES) in the stand-by state in ACT 9 and ACT 10, the processing of the processor 11 shifts to ACT 13 from ACT 9. In ACT 13, the processor 11 increments the number counter X by “1”. Then, the processor 11 sets the obtained exercise code C and the number of exercise repetitions N in association with the table number No. X in the setting table 133 (FIG. 4).

Then, in ACT 15, the processor 11 checks whether or not the number counter X has reached a maximum value Xmax of the table number No. X. For example, in a case where the maximum value Xmax of the table number No. X is “3” as shown in FIG. 4, the processor 11 checks whether or not the number counter X has reached “3”. In a case where the number counter X has not reached the maximum value Xmax of the table number No. X (ACT 15: NO), the processing of the processor 11 returns to ACT 4. Then, the processor 11 executes the processing after ACT 4 in a manner similar to that described above.

In this regard, in a case where the number counter X has reached the maximum value Xmax of the table number No. X (ACT 15: YES), the processing of the processor 11 shifts to ACT 16 from ACT 15. In this manner, the processor 11 stands by until an exercise is selected. Then, in a case where the “end settings” button BTc is input (ACT 5: YES) or the number counter X has reached the maximum value Xmax of the table number No. X (ACT 15: YES) in the stand-by state, the processing of the processor 11 shifts to ACT 16. That is, in a case where the exercise code C and the number of exercise repetitions N are set with respect to the table number No. 1 or each of the table numbers Nos. 1 and No. 2 and an operation input is made on the “end settings” button BTc (ACT 5: YES) or the exercise code C and the number of exercise repetitions N are set to each of the table numbers Nos. 1, 2, and 3, the processing of the processor 11 shifts to ACT 16.

In ACT 16, the processor 11 switches the screen of the touch panel 102 to the confirmation screen SCb (FIG. 8). As shown in FIG. 8, the confirmation screen SCb displays a correspondence table Li of the exercise names of the exercise codes C and the numbers of exercise repetitions N that have been set to the setting table 133. Moreover, software keys of a “register” button BTd and a “cancel” button BTe are arranged in the confirmation screen SCb.

FIG. 8 shows the confirmation screen SCb in a case where settings are made so that the user can be authenticated by the user doing “push-ups” as the first exercise of the exercises, “squats” as the second exercise, and “abdominal crunches” as the third exercise ten times each. In a case where such settings have no problem, the user makes an operation input on the “register” button BTd. In order to modify the settings, the user inputs the “cancel” button BTe.

The description to FIG. 5 will be continued. In ACT 17, the processor 11 that has displayed the confirmation screen SCb stands by until the “register” button BTd is input or the “cancel” button BTe is input. In the stand-by state, in a case where the “register” button BTd has been input (ACT 17: YES), the processing of the processor 11 shifts to ACT 18 from ACT 17. In ACT 18, the processor 11 stores the setting table 133 (FIG. 4) in the external storage 13. Then, the processor 11 terminates the information processing when the setting mode has been selected.

On the other hand, in a case where the “cancel” button BTe has been input (ACT 17: NO), the processing of the processor 11 skips ACT 18. That is, the processor 11 does not store the setting table 133. Then, the processor 11 terminates the information processing when the setting mode has been selected. Here, the processor 11 achieves the function as the setting means 111 by the processing of ACT 1 to ACT 18.

When the user of the smartphone 10 that has stored the setting table 133 (FIG. 4) selects the authentication mode from the menu screen of the user authentication APL 131, the processor 11 starts the information processing of the procedure shown in FIG. 6. In ACT 21, the processor 11 resets the number counter X to “0”. Subsequently, in ACT 22, the processor 11 starts the camera 103. The started camera 103 captures an image of the front or back side of the smartphone 10.

The processing of the processor 11 that has started the camera 103 shifts to ACT 23. In ACT 23, the processor 11 increments the number counter X by “1”. Then, in ACT 24, the processor 11 obtains an exercise code C of the table number No. X and the number of exercise repetitions N from the setting table 133 (FIG. 4).

In ACT 25, the processor 11 checks whether or not the exercise code C is “Null”. In a case where the number counter X is “1”, i.e., the exercise code C of the table number No. 1 is not “Null” (ACT: NO), the processing of the processor 11 shifts to ACT 26 from ACT 25. In ACT 26, the processor 11 activates the determination model (see FIG. 3) associated with the exercise code C. For example, in a case where the exercise code C is “01”, the processor 11 activates a determination model MODa for the exercise “push-ups”. In a case where the exercise code C is “02”, the processor 11 activates a determination model MODb for the exercise “squats”. In a case where the exercise code C is “03”, the processor 11 activates a determination model MODc for the exercise “abdominal crunches”.

The processing of the processor 11 that has activated any one of the determination models shifts to ACT 27. In ACT 27, the processor 11 resets a number-of-repetitions counter n to “0”. Then, in ACT 28, the processor 11 loads the image captured by the camera 103. Then, the processor 11 inputs the loaded captured image to the determination model and obtains a determination result.

For example, the determination model MODa determines whether or not the user has done push-ups on the basis of movements of the parts of the user, such as head, shoulders, elbows, wrists, waist, knees, and ankles shown in the captured image. In a case where the movement of push-ups has been recognized, the determination model MODa transmits a recognition success to the processor 11. In a case where the movement of push-ups has not been recognized, the determination model MODa transmits a recognition failure to the processor 11.

For example, the determination model MODb determines whether or not the user has done squats on the basis of movements of the parts of the user, such as head, shoulders, elbows, wrists, waist, knees, and ankles that are shown in the captured image. In a case where the movement of squats has been recognized, the determination model MODb transmits a recognition success to the processor 11. In a case where the movement of squats has not been recognized, the determination model MODb transmits a recognition failure to the processor 11.

For example, the determination model MODc determines whether or not the user has done abdominal crunches on the basis of movements of the parts of the user, such as head, shoulders, elbows, wrists, waist, knees, and ankles that are shown in the captured image. In a case where the movement of abdominal crunches has been recognized, the determination model MODc transmits a recognition success to the processor 11. In a case where the movement of abdominal crunches has not been recognized, the determination model MODc transmits a recognition failure to the processor 11.

The processing of the processor 11 that has inputted the captured image of the camera 103 to the determination model shifts to ACT 29. In ACT 29, the processor 11 checks whether or not the acknowledge of a recognition success has been received from the determination model. In a case where the acknowledge of a recognition success has not been received (ACT 29: NO), in ACT 30, the processor 11 checks whether or not the acknowledge of a recognition failure has been received from the determination model. In a case where the acknowledge of a recognition failure has also not been received (ACT 30: NO), the processing of the processor 11 returns to ACT 28. In this manner, the processor 11 loads an image captured by the camera 103 and inputs the captured image to the determination model until the acknowledge of a recognition success or recognition failure is received from the determination model. Then, the processor 11 waits for a determination result of the determination model.

In a case where the acknowledge of a recognition success has been received from the determination model (ACT 29: YES), the processing of the processor 11 shifts to ACT 31 from ACT 29. In ACT 31, the processor 11 increments the number-of-repetitions counter n by “1”. Then, in ACT 32, the processor 11 checks whether or not the number-of-repetitions counter n has reached the number of exercise repetitions N associated with the table number No. X.

In a case where the number-of-repetitions counter n has not reached the number of exercise repetitions N (ACT 32: NO), the processing of the processor 11 returns to ACT 28 from ACT 32. The processor 11 executes the processing after ACT 28 in a manner similar to that described above.

Therefore, for example, in a case where with respect to the table number No. 1, “01” is set as the exercise code C and “10” is set as the number of exercise repetitions N, the processor 11 repeats the processing of ACT 28 to ACT 32 until the determination model MODa recognizes the movement of push-ups ten times consecutively from the captured image of the camera 103.

In a case where the number-of-repetitions counter n has reached the number of exercise repetitions N (ACT 32: YES), the processing of the processor 11 shifts to ACT 33 from ACT 32. In ACT 33, the processor 11 checks whether or not the number counter X has reached the maximum value Xmax of the table number No. X. In a case where the number counter X has not reached the maximum value Xmax of the table number No. X (ACT 33: YES), the processing of the processor 11 returns to ACT 23 from ACT 33. In ACT 23, the processor 11 further increments the number counter X by “1”. Therefore, the number counter X is an integer equal to or larger than “2”. In a case where the number counter X is equal to or larger than “2”, i.e., it is the table number No. 2 or the table number No. 3, the exercise code C can be “Null”. In a case where the exercise code C of the table number No. X is “Null” (ACT 25: YES), the processing of the processor 11 shifts to ACT 34 from ACT 25. The processing of ACT 34 will be described later.

On the other hand, in a case where the exercise code C of the table number No. X is not “Null” (ACT 25: NO), the processor 11 executes the processing after ACT 26 in a manner similar to that described above. Therefore, for example, in a case where with respect to the table number No. 2, “02” is set as the exercise code C and “10” is set the number of exercise repetitions N, the determination model MODb for the exercise “squats” is validated. Then, the processor 11 repeats the processing of ACT 28 to ACT 32 until this determination model MODb recognizes the movement of squats ten times consecutively from the captured image of the camera 103.

In a case where the movement of squats has been recognized ten times consecutively (ACT 32: YES), the processing of the processor 11 shifts to ACT 33 from ACT 32. In ACT 33, the processor 11 checks whether or not the number counter X has reached the maximum value Xmax of the table number No. X. In a case where the number counter X has not reached the maximum value Xmax of the table number No. X (ACT 33: NO), the processor 11 returns to ACT 23 from ACT 33. The processor 11 executes the processing after ACT 23 in a manner similar to that described above.

For example, in a case where with respect to the table number No. 3, “03” is set as the exercise code C and “10” is set as the number of exercise repetitions N, the determination model MODc for the exercise “abdominal crunches” is validated. Then, the processor 11 repeats the processing of ACT 28 to ACT 32 until this determination model MODc recognizes the movement of abdominal crunches ten times consecutively from the captured image of the camera 103.

In a case where the movement of abdominal crunches has been recognized ten times consecutively (ACT 32: YES), the processing of the processor 11 shifts to ACT 33 from ACT 32. In ACT 33, the processor 11 checks whether or not the number counter X has reached the maximum value Xmax of the table number No. X. In this manner, the number counter X has reached the maximum value Xmax of the table number No. X (ACT 33: YES), the processing of the processor 11 shifts to ACT 34 from ACT 33.

In this manner, in a case where the processor 11 has detected that the exercise code C of the table number No. X is “Null” (ACT 25: YES) or the number counter X has reached the maximum value Xmax of the table number No. X (ACT 33: YES), the processing of the processor 11 shifts to ACT 34. In ACT 34, the processor 11 recognizes that the user has been authenticated.

That is, for example, in a case where the exercise code C and the number of exercise repetitions N have been set only with respect to the table number No. 1, the processor 11 recognizes that the user has been authenticated by the user repeating the exercise of the exercise identified by the exercise code C in the imaging area of the camera 103 N-times. For example, in a case where the exercise code C and the number of exercise repetitions N of the first exercise have been set with respect to the table number No. 1 and the exercise code C and the number of exercise repetitions N of the second exercise have been set with respect to the table number No. 2, the user first repeats the exercise of the exercise identified by the exercise code C of the first exercise in the imaging area of the camera 103 N-times. Subsequently, the user repeats the exercise of the exercise identified by the exercise code C of the second exercise N-times. Accordingly, the processor 11 recognizes that the user has been authenticated. For example, the same applies to a case where the exercise codes C and the numbers of exercise repetitions N of the three exercises have been set with respect to the table numbers Nos. 1 to 3.

In a case where the user has been authenticated in this manner, the processor 11 unlocks the locked folder 134. As a result, the user is allowed to use the application software stored in the locked folder 134.

It should be noted that in a case where the acknowledge of a recognition failure has been received from the determination model (see FIG. 3) (ACT 30: YES), the processing of the processor 11 shifts to ACT 35 from ACT 30. In ACT 35, the processor 11 recognizes that the user has been rejected. In a case where the user has been rejected, the locked folder 134 is not unlocked. Therefore, the user is not allowed to use the application software stored in the locked folder 134.

Then, the processor 11 terminates the information processing when the authentication mode has been selected. Here, the processor 11 achieves the function as the detecting means 112 by the processing of ACT 26 to ACT 30. The processor 11 achieves the function as the counting means 113 by the processing of ACT 31. The processor 11 achieves the function as the authentication means 114 by the processing of ACT 25 and ACT 32 to ACT 34.

As it will be obvious from the above description, the user of the smartphone 10 essentially has to do a predetermined number of repetitions of the set exercise in order to use the application software in the locked folder 134 that requires user authentication. Therefore, the user can habitually do proper exercises for user authentication. Thus, health problems such as waist pain, neck pain, and eye pain will be reduced.

In addition, the user can arbitrarily set the number of exercises and the number of repetitions of each exercise. Therefore, an exercise that applies optimal physical load on the user can be set so that the user can habitually do it.

### Second Embodiment

The second embodiment uses a multifunction peripheral (MFP) as an example of an electronic apparatus that requires user authentication for using its function. In general, the MFP is placed in a work space and a plurality of users shares the MFP. Therefore, each user owns an ID card and causes the MFP to read the ID card so that each user is allowed to use functions such as printer, FAX, and copying. In view of this, in the second embodiment, the user is allowed to use the MFP functions when the user has been authenticated by exercising in the front of the MFP after the user causes the MFP to read the ID card. In this manner, the MFP according to the second embodiment enables the user to habitually do proper exercises for user authentication.

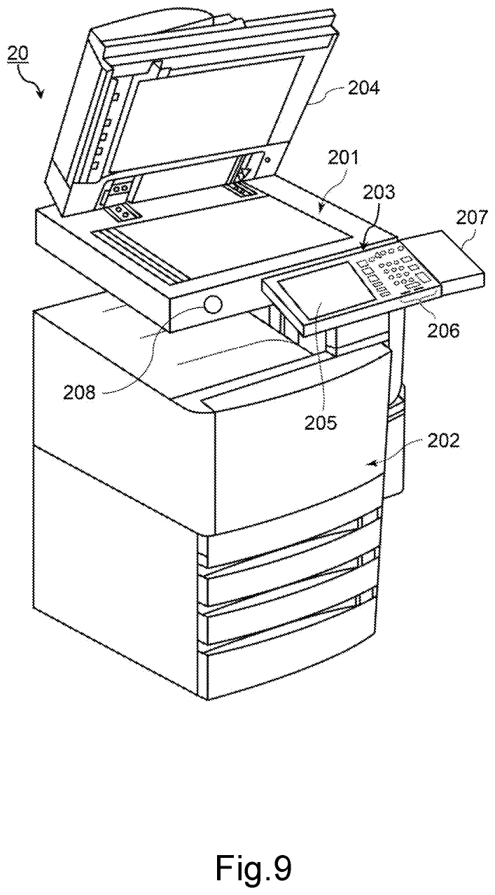

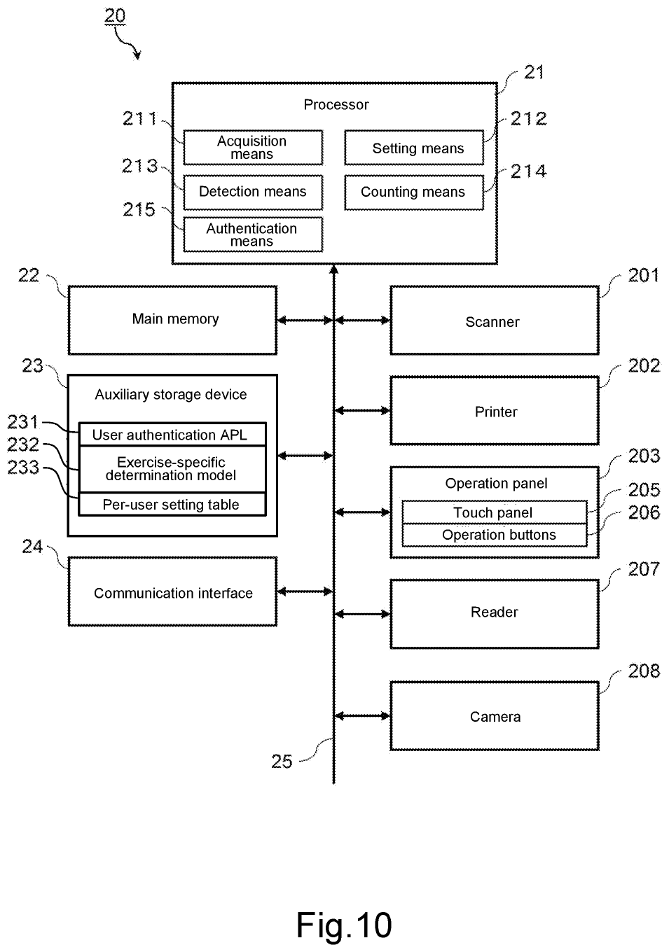

FIG. 9 is a schematic view showing the outer appearance of an MFP 20. FIG. 10 is a block diagram showing a main hardware configuration of the MFP 20. As shown in FIG. 9, the MFP 20 includes a scanner 201, a printer 202, and an operation panel 203.

The scanner 201 is disposed in an upper portion of the main body of the MFP 20. The scanner 201 is a device that optically reads an image of a document. The scanner 201 has a document table glass on which the document to be scanned is placed. The scanner 201 scans the document placed on the document table glass. The scanner 201 has a carriage, a photoelectric conversion unit, and the like. The carriage, the photoelectric conversion unit, and the like are disposed below the document table glass. The scanner 201 reads an image of the entire document by obtaining image data in a main scanning direction through the photoelectric conversion unit while moving the carriage in a sub-scanning direction.

An auto document feeder (ADF) 204 is provided above the scanner 201. The ADF 204 is arranged to be openable/closable with respect to the document table glass. The ADF 204 also functions as a document table cover of the scanner 201. The ADF 204 covers the entire document reading area of the document table glass when the ADF 204 is closed. Moreover, the ADF 204 has a sheet feeding tray, a conveying system, and the like. The ADF 204 in the closed state picks up documents set in the sheet feeding tray one by one and conveys the document so that a reading surface of the picked up document passes through a predetermined reading position. In a case where the ADF 204 conveys the document, the scanner 201 reads an image of the entire document by reading the document surface when the document conveyed to the ADF 204 passes through the predetermined reading position.

The printer 202 has a sheet feeding cassette, a conveying system, and an image forming mechanism. The sheet feeding cassette stores sheets as image forming target media on which images are printed. For example, the sheet feeding cassette is attachable/detachable from a lower portion of the main body of the MFP. A sheet feeding roller picks up the sheets stored in the sheet feeding cassette one by one. The conveying system conveys the sheet that the sheet feeding roller picks up from the sheet feeding cassette to an image forming position or image transferring position.

The image forming mechanism of the printer 202 is a mechanism that forms an image. Various types of image forming can be applied to the image forming mechanism. For example, the image forming mechanism may be an electrophotographic process type, may be an inkjet type, or may be a heat transfer type. Moreover, the image forming mechanism may form a color image or may form a monochrome image. The image forming mechanism forms an image on a sheet fed by the conveying system. Moreover, the image forming mechanism may be one that transfers an image formed on an intermediate transferring member to the sheet supplied by the conveying system at an image transferring position.

The operation panel 203 is a user interface. The operation panel 203 has a touch panel 205 and operation buttons 206. The touch panel 205 displays operation guide, pre-set contents, and the like. Moreover, the touch panel 205 displays icons and the like as the operation buttons. The touch panel 205 detects a site on the display screen, which the user touches. The operation buttons 206 includes a ten key, operation buttons for instructing particular operations, and the like. The user inputs operation instructions through the operation buttons 206 or the touch panel 205 of the operation panel 203.

A reader 207 is attached to the operation panel 203 of the MFP 20. The reader 207 is a device that reads data on a contactless IC card. The contactless IC card is an ID card owned by each user working in the work space where the MFP 20 is placed. The ID card has a unique user ID recorded, which is set to each user in order to identify the user who owns the ID card.

The MFP 20 includes a camera 208. The camera 208 is an image capturing device. For example, a CCD camera is used as the camera 208. The camera 208 captures a still image or moving image in an imaging area, which is a front side of the main body of the MFP, i.e., a side on which the operation panel 203 is provided.

As shown in FIG. 10, the MFP 20 includes a processor 21, a main memory 22, an auxiliary storage device 23, and a communication interface 24. In the MFP 20, the processor 21, the main memory 22, the auxiliary storage device 23, and the communication interface 24 are connected to one another through a system communication channel 25. The system communication channel 25 includes an address bus, a data bus, a control signal line, and the like. The MFP 20 configures a computer by connecting the processor 21, the main memory 22, the auxiliary storage device 23, and the communication interface 24 to one another through the system communication channel 25. Then, in the MFP 20, the scanner 201, the printer 202, the operation panel 203, the reader 207, and the camera 208 are connected to the computer through the system communication channel 25. It is needless to say that devices mounted on the MFP 20 are not limited to the scanner 201, the printer 202, the operation panel 203, the reader 207, and the camera 208.

The processor 21 corresponds to a central processing portion of the computer. The processor 21 controls the respective units in accordance with an operating system or application software in order to achieve various functions as the MFP 20. The processor 21 is a CPU, for example.

The main memory 22 corresponds to a main storage portion of the above-mentioned computer. The main memory 22 includes a nonvolatile memory area and a volatile memory area. The main memory 22 stores an operating system or application software in a nonvolatile memory area. The main memory 22 is capable of storing data required for the processor 21 to execute processing for controlling the respective units in a nonvolatile or volatile memory area. The volatile memory area of the main memory 22 is used as a work area where processor 21 rewrites data as appropriate. The non-volatile memory area is, for example, a read only memory (ROM). The volatile memory area is, for example, a random access memory (RAM).

The auxiliary storage device 23 corresponds to an auxiliary storage portion of the above-mentioned computer. For example, an electric erasable programmable read-only memory (EEPROM), a hard disk drive (HDD), or a solid state drive (SSD) can be the auxiliary storage device 23. The auxiliary storage device 23 stores data used by the processor 21 for performing various types of processing, data generated as a result of processing of the processor 21, and the like. The auxiliary storage device 23 is capable of storing the above-mentioned application software.

The communication interface 24 performs data communication with another computer connected via a network. The other computer is, for example, one or more personal computers placed in the work space. The communication interface 24 receives printing data from the other computer, for example, via the network.

The communication interface 24 sends the data read by the scanner 201, for example, to the other computer via the network.

In the present embodiment, a user authentication APL 231 is installed in the MFP 20 having the above-mentioned configuration. The user authentication APL 231 is application software for user authentication. The user authentication APL 231 may be installed in the main memory 22 or may be installed in the auxiliary storage device 23. FIG. 10 shows a case where the user authentication APL 231 is installed in the auxiliary storage device 23. A method of installing the user authentication APL 231 in the MFP 20 is not particularly limited. The user authentication APL 231 can be installed in the main memory 22 or the auxiliary storage device 23 of the MFP 20 by recording the user authentication APL 231 on a removable recording medium or delivering the user authentication APL 231 by communication via the network. The recording medium can take any form, e.g., a CD-ROM or memory card as long as the recording medium can store a program and a device can read the program from recording medium.

The user authentication APL 231 is application software for detecting a posture of the user doing an exercise from an image captured by the camera 208, counting the number of repetitions the determined exercise has been repeated on the basis of the posture of the user, and authenticating the user of the MFP 20 under the condition that the number of repetitions has reached a set number of repetitions equal to or larger than 2. When the user authentication APL 231 has authenticated the user, the functions such as the printer, FAX, and copying are allowed to be used in the MFP 20.

In order to achieve such functions, the user authentication APL 231 is installed in the MFP 20, such that storage areas of an exercise-specific determination model 232 and a per-user setting table 233 are formed in the auxiliary storage device 23.

The exercise-specific determination model 232 is similar to the exercise-specific determination model 132 (FIG. 3) described in the first embodiment. Therefore, the description is omitted here.

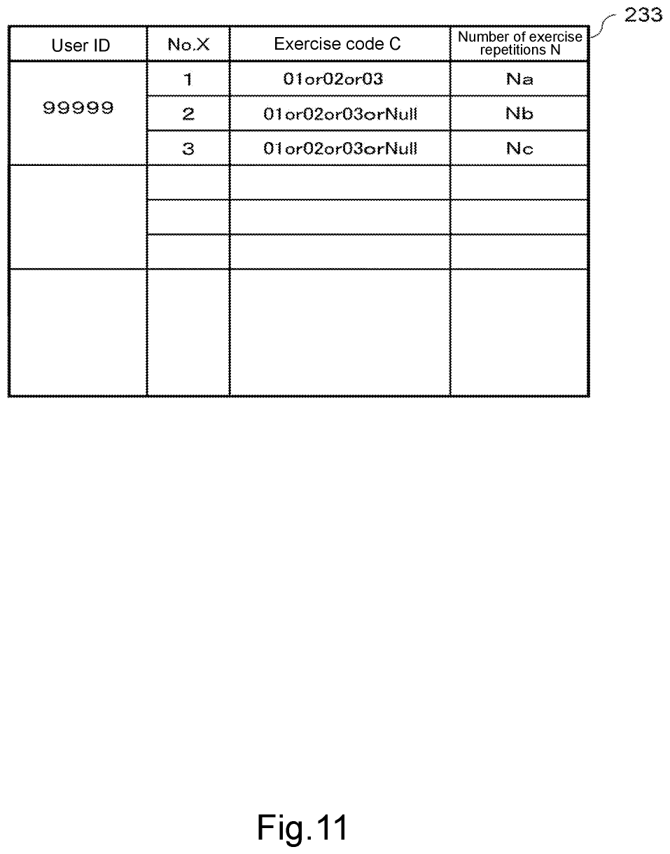

The per-user setting table 233 is an area for setting the exercise code C and the number of exercise repetitions N in the order of a sequence of table numbers Nos. X in association with the user ID of each user as shown in FIG. 11. The table number No. X, the exercise code C, and the number of repetitions N are similar to those of the setting table 133 (FIG. 4) in the first embodiment. That is, the per-user setting table 233 has the setting tables 133 provided for each user of the MFP 20.

Icons of the user authentication APL 231 are displayed on the screen of the touch panel 205 by installing the user authentication APL 231 in the MFP 20. When the user of the MFP 20 makes a touch operation on this icon, the user authentication APL 231 starts. With the start of the user authentication APL 231, the processor 21 achieves functions as an acquisition means 211, a setting means 212, a detecting means 213, a counting means 214, and an authentication means 215 as shown in FIG. 10.

The acquisition means 211 is a function of obtaining identification information for identifying the user. In the present embodiment, the identification information is the user ID. The acquisition means 211 obtains the user ID recorded on the ID card through the reader 207. The setting means 212 is a function of setting to the per-user setting table 233 the exercise code C and the number of exercise repetitions N in the order of the table numbers Nos. X for each user ID (FIG. 11). The detecting means 213 is a function of detecting a posture of the user doing an exercise in the front of the MFP 20 by using the exercise-specific determination model 232 (see FIG. 3). The counting means 214 is a function of counting the number of repetitions the determined exercise has been repeated on the basis of the posture of the user. The authentication means 215 is a function of authenticating the user under the condition that the number of repetitions has reached a set number of repetitions equal to or larger than 2.

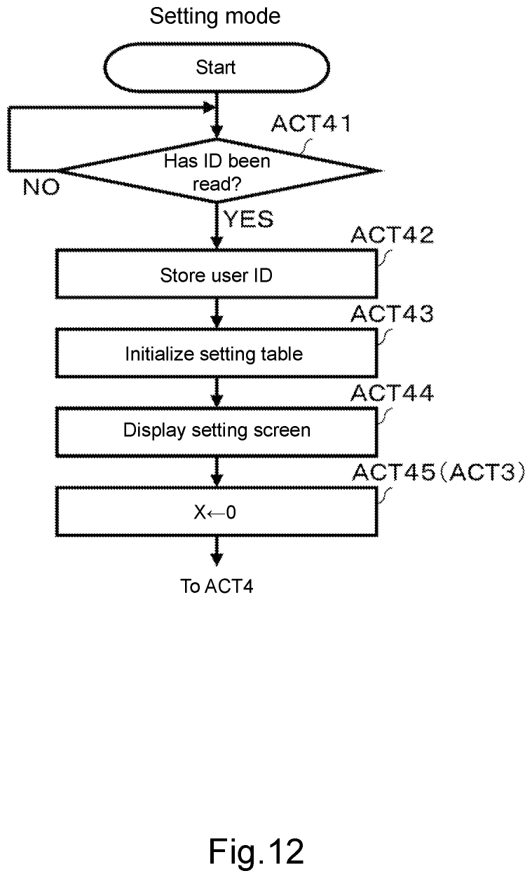

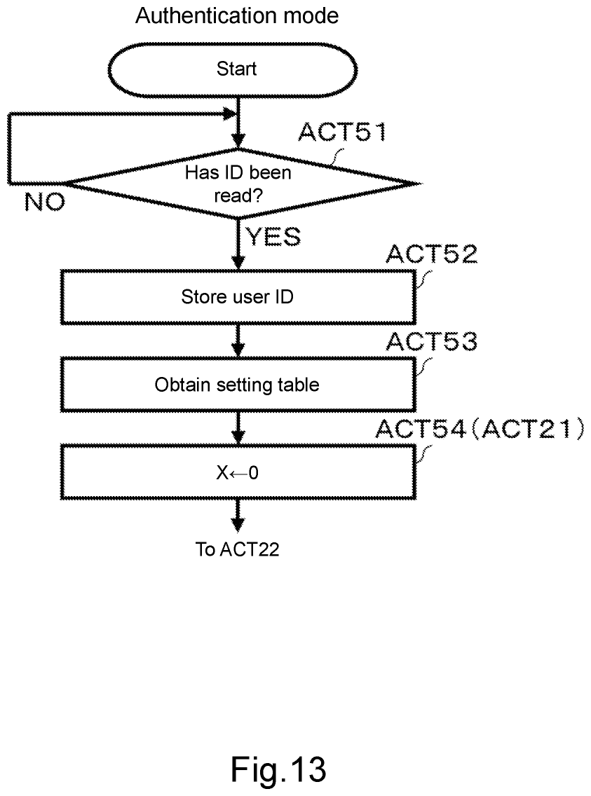

The setting means 212 is a function achieved by the processor 21 operating on a setting mode of the user authentication APL 231. The detecting means 213, the counting means 214, and the authentication means 215 are functions achieved by the processor 11 operating on the authentication mode of the user authentication APL 131. It should be noted that the acquisition means 211 is a function achieved on both the setting mode and the authentication mode. Next, principal operations of the processor 21 will be described with reference to FIGS. 12 and 13.

FIG. 12 is a flowchart showing information processing when the processor 21 operates on the setting mode of the user authentication APL 231. In FIG. 12, processing steps identical to those of FIG. 5 showing the processing according to the first embodiment are denoted by the same reference signs. FIG. 13 is a flowchart showing information processing when the processor 21 operates on the authentication mode of the user authentication APL 231. In FIG. 13, processing steps identical to those of FIG. 6 showing the processing according to the first embodiment are denoted by the same reference signs. It should be noted that the procedure of the information processing shown in each flowchart is an example. The procedure can be modified as appropriate as long as it can provide similar effects.

The user of the MFP 20 starts the user authentication APL 231 by touching one of the icons of the user authentication APL 231 displayed on the touch panel 205. Then, a menu screen of the user authentication APL 231 is displayed on the touch panel 205. Next, the user selects a setting mode from the menu screen. Then, the processor 21 starts the information processing of the procedure shown in the flowchart of FIG. 12.

In ACT 41, the processor 21 stands by until the user ID is read. The user who has selected the setting mode directs the ID card to the reader 207. Accordingly, the reader 207 reads the user ID recorded on the ID card. When the user ID is read (ACT 41: YES), the processing of the processor 21 shifts to ACT 42 from ACT 41. In ACT 42, the processor 21 stores the user ID in the auxiliary storage device 23.

Subsequently, in ACT 43, the processor 21 initializes the per-user setting table 233 (FIG. 11). This initialization clears the exercise code C and the number of exercise repetitions N described in the per-user setting table 233 in association with the stored user ID. As a result, the exercise codes C of the table numbers Nos. X associated with the stored user ID are “Null”. The numbers of exercise repetitions N are “0”.

The processing of the processor 21 that initialized the per-user setting table 233 shifts to ACT 44. In ACT 44, the processor 21 switches the screen of the touch panel 205 to the setting screen. The setting screen is similar to the setting screen SCa (FIG. 7) according to the first embodiment. That is, a pull-down box INa for selecting one of the exercises and an input box INb of the number of exercise repetitions N are arranged on the setting screen. Moreover, software keys of an “execute” button BTa, a “cancel” button BTb, and an “end settings” button BTc are arranged on the setting screen.

The user that has confirmed the setting screen selects one exercise of the exercises “push-ups”, “squats”, and “abdominal crunches” displayed on the pull-down box INa as in the first embodiment. Then, the user inputs the number of repetitions equal to or larger than “2” since a ten key is displayed by touching the input box INb.

In ACT 45, the processor 11 that has displayed the setting screen resets the number counter X to “0”. This processing is similar to the processing of ACT 3 described in the first embodiment. After that, the processor 21 executes the processing of ACT 4 to 18 described in the first embodiment in a manner similar to that described above.

In this manner, in the per-user setting table 233 (FIG. 11), the exercise code C and the number of exercise repetitions N, which have been set by the user identified by the user ID, are described in the order of the table numbers Nos. X in association with the user ID of the ID card read by the reader 207. Here, the processor 21 achieves the function as the acquisition means 211 by the processing of ACT 41 and ACT 42. Moreover, the processor 21 achieves the function as the setting means 212 by the processing of ACT 43, ACT 44, and ACT 4 to ACT 18.

When the user selects the authentication mode from the menu screen of the user authentication APL 231 after the user sets the exercise code C and the number of exercise repetitions N in association with his or her user ID in the per-user setting table 233 (FIG. 11), the processor 21 starts the information processing of the procedure shown in FIG. 13. In ACT 51, the processor 21 stands by until the user ID is read. The user who has selected the authentication mode directs the ID card to the reader 207. Accordingly, the reader 207 reads the user ID recorded on the ID card. When the user ID is read (ACT 51: YES), the processing of the processor 21 shifts to ACT 52 from ACT 51. In ACT 52, the processor 21 stores the user ID in the auxiliary storage device 23.

Subsequently, in ACT 53, the processor 21 obtains data described in association with the stored user ID, from the per-user setting table 233 (FIG. 11). That is, the processor 21 obtains an exercise code C and the number of exercise repetitions N described in the order of the table numbers Nos. X. Then, in ACT 54, the processor 21 resets the number counter X to “0”. This processing is similar to the processing of ACT 21 described in the first embodiment. After that, the processor 21 executes the processing of ACT 21 to 35 described in the first embodiment in a manner similar to that described above.

In this manner, the processor 21 recognizes that the user has been authenticated by the user repeating the exercise determined by the exercise code C in the imaging area of the camera 208 provided in the MFP 20 N-times (the number of exercise repetitions N). As a result, the user is allowed to use the functions of the MFP 20 such as the printer, FAX, and copying.

Here, the processor 21 achieves the function as the acquisition means 211 by the processing of ACT 51 and ACT 52. Moreover, the processor 21 achieves the function as the detecting means 112 by the processing of ACT 26 to ACT 30, achieves the function as the counting means 113 by the processing of ACT 31, and achieves the function as the authentication means 114 by the processing of ACT 25 and ACT 32 to ACT 34 as in the first embodiment.

As described above in detail, in accordance with the second embodiment, every user of the MFP 20 essentially has to do a predetermined number of repetitions of the set exercise in order to use the functions such as the printer, FAX, and copying. Therefore, each user can habitually do proper exercises for user authentication. Thus, health problems such as waist pain, neck pain, and eye pain of each user working in the work space will be reduced.

In addition, each user can arbitrarily set the number of exercises and the number of repetitions of each exercise. Therefore, an exercise that applies optimal physical load on the user can be set so that the user can habitually do it.

### Other Embodiments

In each of the above-mentioned embodiments, the plurality of exercises can be set. In another embodiment, only one exercise may be set. In this case, the user sets only the number of exercise repetitions N on the setting screen SCa (FIG. 7). It should be noted that the exercises are not limited to the three exercises of “push-ups”, “squats”, and “abdominal crunches”. For example, “jumps”, “sideway jumps”, “leg lifts”, “arm circles” can apply appropriate physical load on the user, so the user can set them as the exercises.

In each of the above-mentioned embodiments, the case where the posture of the user doing the exercise is detected on the basis of the image captured by the camera 103 or the camera 208 has been described as an example. The means for detecting the posture of the user doing the exercise is not limited thereto. For example, in a case of detecting the exercise “jumps”, a sheet-like pressure sensor may be provided near an electronic apparatus that requires user authentication. Then, the user repeats jumps on the pressure sensor. The electronic apparatus may receive an output signal from the pressure sensor, which fluctuates depending on a posture of the user at that time, and detect the posture of the user doing the exercise.

In each of the above-mentioned embodiments, the smartphone 10 and the MFP 20 have been described as examples of the electronic apparatus that requires user authentication for using its function. It is needless to say that the electronic apparatus is not limited to the smartphone 10 and the MFP 20. For example, the first embodiment can be applied to an electronic apparatus personally used by an individual user, such as a tablet terminal and a PC, as it is. On the other hand, the second embodiment can be applied to an electronic apparatus shared by a plurality of users, such as a copying machine, a scanner, and a shared PC, as it is.

In each of the above-mentioned embodiments, for example, in a case where the number of exercise repetitions N has been set to each exercise of the three exercises, the user can be authenticated only when the number of exercise repetitions N for each exercise has been recognized in the order of the set three exercises. In this regard, for example, a configuration in which the user can be authenticated when an exercise of any one of the exercises has been done N-times (the number of exercise repetitions N) has been recognized may be employed. Such a configuration enables the user to change the exercise for user authentication as appropriate without modifying the settings of the setting table 133 or the per-user setting table 233. This configuration can enhance the effect that the user can habitually do proper exercises because the user is not bored with it.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic apparatus that requires authentication of a user, comprising:
a camera that images the user;
a storage that stores a determination model, the determination model being a machine learning algorithm that receives input of a moving image of the user captured by the camera, detects parts of a body of the user on a basis of the moving image, and determines an exercise of the user on a basis of movements of the parts; and
the processor configured to
load the moving image captured by the camera,
detect a posture of the user doing the exercise by inputting the loaded captured moving image to the determination model and obtaining a determination result,
count the number of repetitions a pre-set exercise has been repeated on a basis of the detected posture of the user, and authenticate the user under a condition that the counted number of repetitions has reached the set number of repetitions.

2. The electronic apparatus according to claim 1, wherein
the storage stores an exercise-specific determination model in which a plurality of exercise codes representing the exercises and the determination model associated with each of the plurality of exercise codes are described, and
the processor
obtains a pre-set exercise code, and
inputs the loaded captured moving image to the determination model associated with the obtained exercise code.

3. The electronic apparatus according to claim 2, wherein
the storage further stores a setting table in which an exercise code for authentication of the user is pre-set, and
the processor obtains the exercise code set in the setting table.

4. The electronic apparatus according to claim 2, wherein
the storage further stores a setting table in which the set number of repetitions for authentication of the user is pre-set, and
the processor
obtains the set number of repetitions set in the setting table, and
authenticates the user under a condition that the counted number of repetitions has reached the obtained set number of repetitions.

5. The electronic apparatus according to claim 2, wherein
the storage further stores a setting table in which the plurality of exercise codes and the set number of repetitions associated with each of the plurality of exercise codes are pre-set, and
the processor
obtains the exercise code and the set number of repetitions associated with the exercise code from the setting table,
detects a posture of the user doing the exercise by inputting the loaded captured moving image to the determination model associated with the obtained exercise code and obtaining a determination result,
counts the number of repetitions the exercise indicated by the obtained exercise code has been repeated on a basis of the detected posture of the user, and
authenticates the user under a condition that the counted number of repetitions has reached the obtained set number of repetitions.

6. The electronic apparatus according to claim 1, further comprising
a touch panel that inputs the set number of repetitions by a touch operation of the user, wherein
the storage further stores a setting table in which the set number of repetitions for authentication of the user is pre-set, and
the processor
obtains, on a setting mode, the set number of repetitions input via the touch panel and stores the obtained set number of repetitions in the setting table, and
obtains, on the authentication mode, the set number of repetitions from the setting table and authenticates the user under a condition that the number of repetitions has reached the obtained set number of repetitions.

7. The electronic apparatus according to claim 1, wherein
the processor
counts the number of repetitions of each exercise of a plurality of pre-set exercises has been repeated for each exercise on a basis of the posture of the user, and
authenticates the user under a condition that each of a plurality of numbers of repetitions, each of which has been counted for each of the plurality of exercises, has reached the numbers of repetitions equal to or larger than 2.

8. The electronic apparatus according to claim 7, further comprising
a touch panel that inputs the exercise code and the set number of repetitions, wherein
the storage further stores
an exercise-specific determination model in which a plurality of exercise codes representing the exercises and the determination model associated with each of the plurality of exercise codes are described, and
a setting table in which the plurality of exercise codes and the set number of repetitions associated with each of the plurality of exercise codes are pre-set, and
the processor
on a setting mode
obtains the exercise code and the set number of repetitions input via the touch panel, and
stores the obtained exercise code and the set number of repetitions in the setting table, and
on an authentication mode
obtains the exercise code and the set number of repetitions from the setting table,
detects a posture of the user doing the exercise by inputting the loaded captured moving image to the determination model associated with the obtained exercise code and obtaining a determination result,
counts the number of repetitions the exercise indicated by the obtained exercise code has been repeated on the basis of the detected posture of the user, and
authenticates the user under a condition that the counted number of repetitions has reached the obtained set number of repetitions.

9. The electronic apparatus according to claim 1, further comprising
a reader that reads identification information of the user, wherein
the storage stores
an exercise-specific determination model in which a plurality of exercise codes representing the exercises and the determination model associated with each of the plurality of exercise codes are described, and
a setting table in which the plurality of exercise codes and the set number of repetitions associated with each of the plurality of exercise codes are pre-set for each identification information of the user, and
the processor
obtains the identification information of the user read through the reader,
obtains the exercise code and the set number of repetitions associated with the obtained identification information of the user from the setting table,
detects a posture of the user doing the exercise by inputting the loaded captured moving image to the determination model associated with the obtained exercise code and obtaining a determination result,
counting the number of repetitions the exercise indicated by the obtained exercise code has been repeated on a basis of the detected posture of the user, and
authenticates the user identified by the identification information of the user under a condition that the counted number of repetitions has reached the obtained set number of repetitions.

10. A control method for an electronic apparatus that requires authentication of a user, comprising:
imaging the user by a camera;
storing, in a storage, a determination model, the determination model being a machine learning algorithm that receives input of a moving image of the user captured by the camera, detects parts of a body of the user on a basis of the moving image, and determines an exercise of the user on a basis of movements of the parts;
loading the moving image captured by the camera;
detecting a posture of the user doing the exercise by inputting the loaded captured moving image to the determination model and obtaining a determination result;
counting the number of repetitions a pre-set exercise has been repeated on a basis of the detected posture of the user; and
authenticating the user under a condition that the counted number of repetitions has reached the set number of repetitions.
